(19) United States
(12) Patent Application Publication
(10) Pub. No.: US 2026/0232253 A1
(43) Pub. Date: Aug. 13, 2026

(54) METHODS FOR DETECTING AND MONITORING NEURAL OSCILLATIONS

(71) Applicant: Cognito Therapeutics, Inc., Cambridge, MA (US)

(72) Inventors: Ralph Kern, Cambridge, MA (US); Mihály Hajòs, Cambridge, MA (US)

(21) Appl. No.: 19/638,569

(22) Filed: Apr. 3, 2026

Related U.S. Application Data
(63) Continuation of application No. PCT/US2024/049952, filed on Oct. 4, 2024.
(60) Provisional application No. 63/588,023, filed on Oct. 5, 2023.

(51) Int. Cl. A61B 5/377; A61B 5/374

(52) U.S. Cl. CPC A61B 5/377; A61B 5/374; A61B 2503/40

(57) ABSTRACT

In some aspects, the present disclosure describes methods of confirming or verifying the administration of a non-invasive sensory stimulus to a subject. In some aspects, further disclosed are devices and systems useful for confirming or verifying the administration of a non-invasive sensory stimulus to a subject.

### CROSS-REFERENCE

This application is a continuation of International Application No. PCT/US2024/049952, filed Oct. 4, 2024, which application claims the benefit of U.S. Provisional Patent Application No. 63/588,023 filed on Oct. 5, 2023, each of which is incorporated by reference in their entirety.

### BACKGROUND

Neural oscillation occurs in humans or animals and includes rhythmic or repetitive neural activity in the central nervous system. Neural tissue can generate oscillatory activity by mechanisms within individual neurons or by interactions between neurons. Oscillations can appear as either oscillations in membrane potential or as rhythmic patterns of action potentials, which can produce oscillatory activation of post-synaptic neurons. Synchronized activity of a group of neurons can give rise to macroscopic oscillations, which can be observed noninvasively by electroencephalography (“EEG”). Neural oscillations can be characterized by their frequency, amplitude, and phase. Neural oscillations can give rise to electrical impulses that form a brainwave. These signal properties can be observed from neural recordings using time-frequency analysis.

### SUMMARY

In some aspects, the present disclosure provides a method of confirming or verifying the administration of a non-invasive sensory stimulus to a subject comprising: (a) administering a non-invasive sensory stimulus to the subject; and (b) quantifying the non-invasive sensory stimulus was administered to the subject, thereby verifying that the non-invasive sensory stimulus was administered to the subject.

In some cases, the non-invasive sensory stimulus has a gamma frequency.

In some cases, the administering the non-invasive sensory stimulus to the brain of the subject results in an alteration of neural activity in the subject.

In some cases, wherein the verifying comprises evaluating the alteration of neural activity non-invasive sensory stimulus in the subject.

In some cases, the evaluating comprises measuring a gamma waveform in the brain of the subject.

In some cases, the measuring comprises neuroimaging.

In some cases, the neuroimaging comprises magnetic resonance imaging (MRI), computer tomography, positron emission tomography (PET) imaging, diffusion imaging, function near-infrared spectroscopy, or any combination thereof. In some cases, the MRI comprises a functional MRI (fMRI).

In some cases, the measuring comprises an electrical readout of neurophysiology.

In some cases, the electrical readout of neurophysiology comprises an electroencephalogram (EEG) recording, magnetoencephalography (MEG) recording, or local field potential.

In some cases, the EEG recording or local field potential measure a feature of the gamma waveform. In some cases, the feature of the gamma waveform comprises the amplitude, the frequency, the duration, the frequency distribution, the maximum frequency, the minimum frequency of the gamma waveform, or any combination thereof.

In some cases, the measuring or perceiving comprises quantifying a therapeutic efficacy in the subject caused by administering the non-invasive sensory stimulus in the subject.

In some cases, the therapeutic efficacy comprises maintenance of brain volume, cortical thickness, synaptic connectivity, cognitive function, sleep quality, myelination, daily living activities, or a combination thereof.

In some cases, the non-invasive sensory stimulus comprises a visual stimulus, an auditory stimulus, a tactile stimulus, a vibrotactile stimulus, a peripheral nerve stimulus, or a combination thereof.

In some cases, the characteristic of the non-invasive sensory stimulus comprises a stimulus feature and a stimulus exposure.

In some cases, the stimulus feature comprises an amplitude or intensity, a frequency, a tone, a color, a luminance, a signal delay, an offset, a duration, a static image, a sinusoidal grating, a sound, a dynamic image, a dynamic sound, or any combination thereof.

In some cases, the frequency of the non-invasive sensory stimulus is from 20 to 100 Hz. In some cases, the frequency of the non-invasive sensory stimulus is 40 Hz.

In some cases, the stimulus exposure comprises a session duration of at least 15 minutes, at least 30 minutes, at least 45 minutes, or at least one hour.

In some cases, the stimulus exposure further comprises a regimen duration of at least one day, one week, one month, three months, six months, nine months, one year, or any combination thereof.

In some cases, the stimulus exposure comprises at least one exposure per day, at least two exposures per day, at least three exposures per day, at least four exposures per day, or at least five exposures per day.

In some cases, the measuring comprises analyzing the brain or a specific brain region of the subject.

In some cases, the specific brain region comprises an entorhinal cortex, a hippocampus, a cerebral cortex, a visual association cortex, an auditory association cortex, a cingulate lobe, an amygdala, a thalamic nucleus, a cingulate lobe, a pons, a brainstem, a cerebellum, a lateral ventricle, an occipital lobe, a parietal lobe, a temporal lobe, a midbrain, a striatum, a basal ganglia, a globus pallidus, a substantia nigra, or any combination thereof.

In some cases, the therapeutic efficacy further comprises a determination that the amplitude, the frequency, the duration, the frequency distribution, the maximum frequency, the minimum frequency of the gamma waveform, or any combination thereof are increased in the subject following administration of the non-invasive sensory stimulus compared to the subject prior to administration.

In some cases, the subject comprises a mammal, a non-human primate, or a human.

In some cases, the method of confirming or verifying the administration of a non-invasive sensory stimulus to a subject is carried out by a neural stimulation system (NSS).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative cases, in which the principles of the disclosure are utilized, and the accompanying drawings of which:

FIG. 1 illustrates a block diagram depicting a system to perform neural stimulation via visual stimulation in accordance with an embodiment.

FIGS. 2A and 2B are block diagrams depicting cases of computing devices useful in connection with the systems and methods described herein.

FIG. 3 shows a schematic of some aspects and parameters characterizing stimulus audio and visual components of non-invasive stimulation as delivered respectively by Audio Stimulus Module (110; FIG. 4) and Visual Stimulus Module (120; FIG. 4) of Stimulus Delivery System (170; FIG. 4). Numbers and relative dimensions of elements in FIG. 3 are adjusted for presentation and may not represent those for actual cases.

FIG. 4 provides a block diagram of an exemplary stimulus delivery system and analysis and monitoring system, said analysis and monitoring system comprising modules specific to sleep-related monitoring and/or analysis.

FIG. 5 shows graphs comparing the relative change in active durations, with the Y-axis indicating change relative to Weeks 1-12 during Weeks 13-24. FIG. 5 demonstrates a reduction in duration of active periods for the treatment group and, consequently, a reduction in sleep fragmentation leading to increased sleep quality. In contrast, the opposite effect was seen with the sham group, which is represented by the line closest to the gray arrow. Panel A of FIG. 5 shows the relative change based on the duration of active periods, and Panel B of FIG. 5 shows the normalized nighttime active durations, calculated by dividing the duration of each active period by the duration of the matching entire nighttime period.

### DETAILED DESCRIPTION

In some aspects, the present disclosure provides, methods, systems, and devices for confirming or verifying the administration of a non-invasive sensory stimulus to a subject.

Methods, devices, and systems that administer a non-invasive sensory stimulus to a subject may be useful in inducing a response within the brain of a subject, for example, by inducing neural oscillations. In some cases, the methods, devices, and systems may be improved by confirming or verifying that the stimulus was administered to the brain of the subject by quantifying the stimulus in the subject, for example, by quantifying an alteration of neural activity in the subject. For example, if the confirmation or verification determines that the non-invasive sensory stimulus was not administered to the brain, was administered to the wrong brain region, or was administered in a manner not consistent with the induction of the response within the brain, an additional stimulus can be applied to induce, and/or modulate the response. In this manner, the confirming or verifying can act as a quality control mechanism for the methods, devices, and systems of administering a non-invasive sensory stimulus to a subject.

In some cases, a method of confirming or verifying the administration of a non-invasive sensory stimulus to a subject comprises administering a non-invasive sensory stimulus to a brain of the subject, for example, a visual, auditory, tactile, vibrotactile, or peripheral nerve stimulus. In some cases, the stimulus has a gamma frequency. Administration of such a stimulus to the brain of a subject can result in an alteration of neural activity in a subject. The methods, devices, and systems of the disclosure are designed to verify, evaluate, and/or measure the alteration of this neural activity to verify the administration of the stimulus to the subject. This measuring can comprise measuring a gamma waveform in the brain of the subject, neuroimaging, neurophysiology, quantification of an alteration that includes a therapeutic response and/or therapeutic efficacy, or any combination thereof.

Methods of diagnosing, treating, evaluating, monitoring, and predicting responses to neurological disorders or conditions using a non-invasive sensory stimulus are also disclosed in PCT/US2023/017909, PCT/US2023/15570, PCT/US2022/44760, PCT/US2022/44755, PCT/US2022/19370, and PCT/US2021/71003, the disclosures of which are hereby incorporated by reference.

### Non-invasive Sensory Stimulus

### Nature of Stimulus (Modality)

In some cases, administering may be a non-invasive procedure. In some cases, administering may be indirect (e.g., without physical contact) stimulation of optical nerves. In some cases, administering may use light. In some cases, administering may be indirect stimulation of auditory nerves. In some cases, administering may be indirect stimulation of any one of the nerves disclosed herein. In some cases, administering may use sound. In some cases, administering may be direct stimulation. In some cases, administering may be electricity delivered to a region in the subject's body. In some cases, administering may be vibration delivered to a region in the subject's body. In some cases, the region in the subject's body may be skin on the head of the subject, surface of the skull of the subject, surface of a membrane surrounding the subject's brain, the subject's brain, a region in the subject's brain, a retinal nerve of the subject, or a cochlear nerve of the subject. In some cases, the non-invasive sensory stimulus may be a visual stimulus, an auditory stimulus, a kinesthetic stimulus, or any combination thereof. In some cases, the gamma oscillation inducing non-invasive sensory stimulus may be a visual stimulus, an auditory stimulus, a kinesthetic stimulus, or any combination thereof. In some cases, the non-invasive sensory stimulus comprises a visual stimulus. In some cases, the subject's eyes are opened during the administration of the visual stimulus. In some cases, the subject's eyes are closed during the administration of the visual stimulus. In some cases, the subject's eyes are closed during the administration of the visual stimulus, and the gamma waveform in the subject can still be detected, adjusted, and/or neuromodulated. In some cases, the subject's eyes are closed during the administration of the visual stimulus, and the alteration of neural activity in the subject can still be detected, adjusted, and/or neuromodulated. In some cases, the non-invasive sensory stimulus comprises an auditory stimulus. In some cases, the non-invasive sensory stimulus comprises a visual stimulus and an auditory stimulus. In some cases, the non-invasive sensory stimulus comprises a visual stimulus and an auditory stimulus, wherein the visual stimulus is synchronous with the auditory stimulus. In some cases, the non-invasive sensory stimulus comprises a visual stimulus and an auditory stimulus, wherein the visual stimulus is asynchronous with the auditory stimulus. The non-invasive sensory stimulus may be provided using any one of the devices disclosed herein. The gamma waveform-inducing non-invasive sensory stimulus may be provided using any one of the devices or methods disclosed herein.

### Stimulus Features

In some cases, at least some of the parameters or characteristics of the non-invasive signal administered to a subject correspond to those specified in one or more of U.S. Pat. No. 10,307,611 B2, U.S. Pat. No. 10,293,177 B2, or U.S. Pat. No. 10,279,192 B2. In some cases, at least some of the parameters or characteristics of the non-invasive signal administered to a subject correspond to those specified in one or more of U.S. Pat. No. 10,159,816 B2 or U.S. Pat. No. 10,265,497 B2.

In some cases, the non-invasive sensory stimulus comprises a stimulus feature. In some cases, the stimulus feature comprises an amplitude or intensity, a frequency, a pulse rate, a tone, a color, a luminance, a signal delay, an offset, a duration, a static image, a sinusoidal grating, a sound, a dynamic image, a dynamic sound, or any combination thereof. In some cases, the stimulus feature comprises a visually perceptive feature including an amplitude or intensity, a color, a luminance, a signal delay, an offset, a duration, a sinusoidal grating, a square-wave grating, a checkerboard pattern, a direction of motion, a color/shade contrast.

### Visual Stimulus

In some cases, a non-invasive sensory stimulus comprises a visual stimulus. In some cases, the visual stimulus comprises a light wave within the range of the visual spectrum. In some cases, the visual stimulus comprises a light wave within the ultraviolet spectrum.

In some cases, the visual stimulus comprises one or more bursts of light waves resembling one or more flashes or pulses of light (e.g., a pulse rate). In some cases, the visual stimulus comprises a pulse rate from about 35 Hz to about 100 Hz. In some cases, the visual stimulus comprises a pulse rate from about 35 Hz to 60 Hz. In some cases, the visual stimulus comprises a pulse rate of 40 Hz.

### Auditory Stimulus

In some cases, a non-invasive sensory stimulus comprises an auditory stimulus. In some cases, the auditory stimulus comprises a sound with amplitude or intensity, a frequency, a pulse rate, a tone, a signal delay, an offset, a duration, a sinusoidal grating, a dynamic sound, a perceived direction of motion, or any combination thereof. In some cases, the auditory stimulus comprises one or more acoustic waves. In some cases, the sound comprises an ultrasound or an infrasound. In some cases, the sound comprises a tone or frequency perceptible to the subject. In some cases, the sound comprises a tone or frequency imperceptible to the subject. In some cases, the sound comprises an acoustic frequency from about 0 Hz to about 50 kHz. In some cases, the sound comprises an acoustic frequency from about 20 Hz to about 20 kHz. In some cases, the sound comprises an acoustic frequency from about 8 Hz to about 12 kHz. In some cases, the sound comprises an acoustic frequency of 10 kHz. In some cases, the dynamic sound comprises a tone or acoustic frequency perceptible to the subject. In some cases, the dynamic sound comprises a tone or acoustic frequency imperceptible to the subject. In some cases, the dynamic sound comprises an acoustic frequency from about 20 Hz to about 20 kHz. In some cases, the auditory stimulus comprises an acoustic frequency capable of modulating a gamma waveform in the subject. In some cases, the acoustic frequency capable of modulating a gamma waveform in the subject comprises an acoustic waveform of 0.1 Hz, 1 Hz, 5 Hz, 10 Hz, 20 Hz, 25 Hz, 30 Hz, 31 Hz, 32 Hz, 33 Hz, 34 Hz, 35 Hz, 36 Hz, 37 Hz, 38 Hz, 39 Hz, 40 Hz, 41 Hz, 42 Hz, 43 Hz, 44 Hz, 45 Hz, 46 Hz, 47 Hz, 48 Hz, 49 Hz, 50 Hz, 60 Hz, 70 Hz, 80 Hz, 90 Hz, 100 Hz, 150 Hz, 200 Hz, 250 Hz, 300 Hz, 400 Hz, 500 Hz, 1000 Hz, 2000 Hz, 3000 Hz, 4,000 Hz, 5000 Hz, 6,000 Hz, 7,000 Hz, 8,000 Hz, 9,000 Hz, or 10,000 Hz.

### Tactile Stimulus

In some cases, a non-invasive sensory stimulus comprises a tactile stimulus. In some cases, a tactile stimulus can be any means that activates somatosensory transduction pathways. In some cases, a tactile stimulus can be generated by a wearable component attached to the user such as with a headband, wristband, piece of clothing, fashion accessory, or other means of coupling physically to the user. In some cases, the tactile stimulus can be generated by any means that activates somatosensory transduction pathways for instance by activating peripheral receptors that mediate touch, temperature sensation, or pain. In various cases, tactile stimuli can be generated by a piezo actuator, buzzer, heating element, or other mechanism known to one skilled in the art. Examples of non-contact tactile stimulus actuators include ultrasound transducers, components that generate a magnetic field, and other non-contact means to activate somatosensory transduction pathways.

### Vibrotactile Stimulus

In some cases, a non-invasive sensory stimulus comprises a vibrotactile stimulus. In some cases, the vibrotactile stimulus can comprise any vibrotactile stimulus or method of delivering a vibrotactile stimulus known in the art. For example, the vibrotactile stimulus can include any means that induces the perception of vibration through touch. In some cases, a vibrotactile stimulus can be any stimulus that activates the somatosensory system. In some cases, a vibrotactile stimulus can include a vibration.

### Peripheral Nerve Stimulus

In some cases, a non-invasive sensory stimulus comprises a peripheral nerve stimulus. In some cases, the peripheral nerve stimulus can comprise any peripheral nerve stimulus or method of delivering a peripheral nerve stimulus known in the art. For example, the peripheral nerve stimulus can include an electrode device or an implanted electrode device capable of electrically stimulating a peripheral nerve.

### Stimulus Features and Features Values

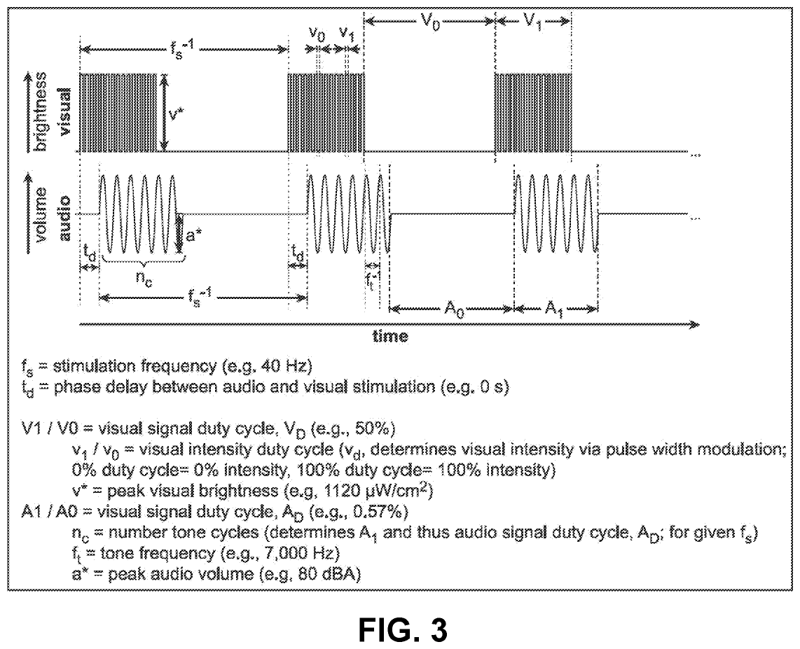

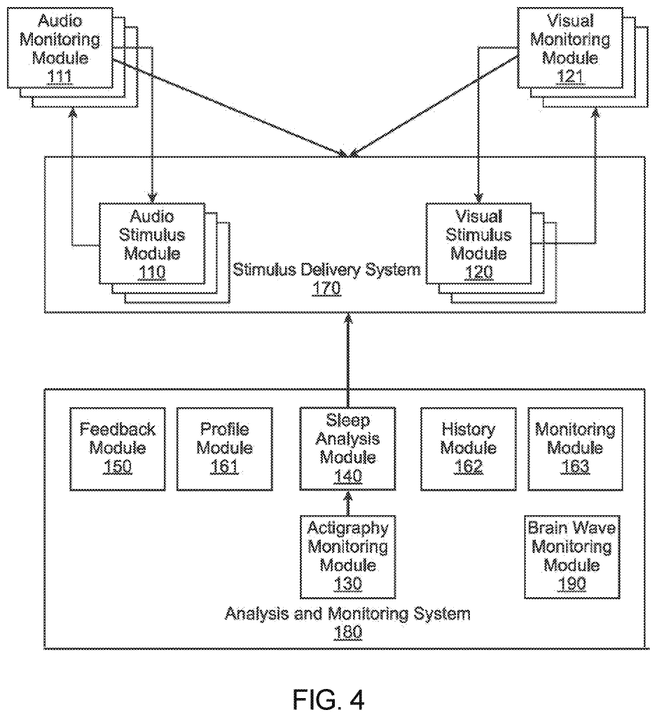

In some cases, gamma oscillation inducing non-invasive sensory stimulus parameters are configured with a stimulus frequency (e.g., fs in FIG. 3) of approximately 30 Hz to approximately 50 Hz for both audio and visual signals. In some cases, audio and visual signals are offset relative to each other by a delay (e.g., td in FIG. 3). In exemplary embodiment audio and visual signals are synchronized (td=0 s).

In some cases, gamma oscillation inducing non-invasive sensory stimulus parameters are configured with a variety of timing and intensity parameters. In an exemplary embodiment, these parameters include those illustrated in FIG. 3. In some cases, these parameters are preconfigured; in some cases they are adjusted at least in part by a third party such as a caregiver or healthcare provider; in some cases one or more parameters are adjusted responsive to measurements or analysis of one or more of: user context, measured sleep quality related parameters associated with the user, observed, or detected use of the stimulation device. In some cases, gamma oscillation inducing non-invasive sensory stimulus parameters are adjusted to detected or analyzed neurogenerative disease symptom progression. Various frequencies and various intensities may be used as parameters for the gamma oscillation inducing non-invasive sensory stimulus.

In some cases, one or more stimulus parameters are based at least partially on various clinical measures of treatment outcomes of cognitive function disclosed herein. In some cases, varying combinations stimulus parameters are used during different time periods and subsequent stimulation parameters are selected at least in part based on comparison of clinical measures of treatment outcomes for cognitive function among at least some of those periods.

In some cases, the present disclosure delivers 40 Hz non-invasive audio, visual, or combined audio-visual stimulation. In some cases, stimulus is delivered at one or more stimulation frequencies (e.g., fs in FIG. 3). In some cases, stimulus is delivered at one or more stimulation frequencies (e.g., fs in FIG. 3) in the approximate range of 30-50 Hz. In some cases, “gamma” refers to frequencies in the range 30-50 Hz. In some cases, the stimulus is periodic. In some embodiment, the stimulus is non-periodic. In some cases, the stimulus is a periodic stimulus with non-periodic components. In some cases, the stimulus is intermittent. In some cases, stimulus is delivered based at least in part on a user's detected, reported, or demographically or individually associated or dominant alpha wave frequency.

In some cases, specific visual parameters include one or more of: stimulation frequency, intensity (brightness), hue, visual patterns, spatial frequency, contrast, and duty-cycle. In an exemplary embodiment, visual stimulation is provided at a stimulation frequency of 40 Hz, brightness between 0 μW/cm2 to 1120 μW/cm2, and 50% visual signal duty-cycle.

In some cases, non-invasive stimulation is delivered as combined visual and auditory stimulation, delivered at 40 Hz frequency. In some cases, visual and auditory stimulation is synchronized to begin each cycle simultaneously. In some cases, the beginning of each auditory and visual stimulation cycle is offset by a configured time. In some cases, visual and auditory signals are delivered at an intensity clearly recognized by subjects and adjusted to their tolerance level.

In some cases, at least some of the parameters or characteristics of the non-invasive signal administered to a subject correspond to those specified in one or more of U.S. Pat. 10,307,611 B2, U.S. Pat. No. 10,293,177 B2, or U.S. Pat. No. 10,279,192 B2. In some cases, at least some of the parameters or characteristics of the non-invasive signal administered to a subject correspond to those specified in one or more of U.S. Pat. No. 10,159,816 B2 or U.S. Pat. No. 10,265,497 B2.

In some cases, specific audio parameters include one or more of: stimulation frequency, intensity (volume), and duty-cycle. In some cases, audio frequency is adjusted responsive to a subject's hearing characteristics, for example to frequencies that a subject is better at hearing. In an exemplary embodiment, audio stimulation is provided at an audio tone frequency of 7,000 Hz, volume level between 0 dBA to 80 dBA, and 0.57% audio signal duty-cycle.

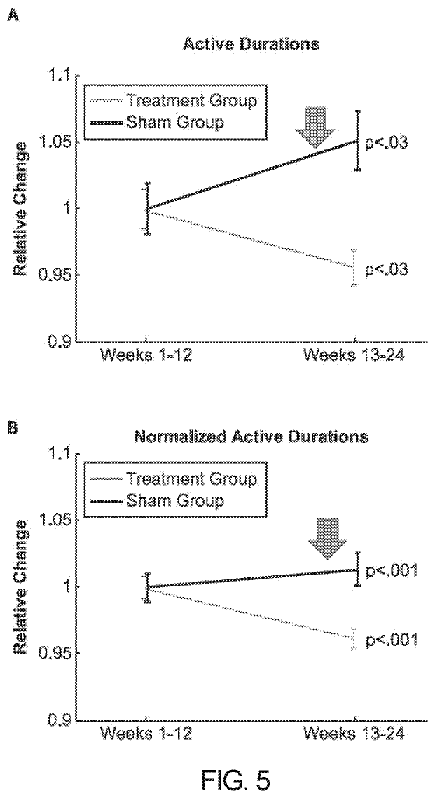

In some cases, non-invasive stimulation parameters are selected directed at evoking gamma wave oscillations in the brains of human subjects. In some cases, non-invasive stimulation parameters are selected directed at inducing alpha waves in human subjects (FIG. 5). In some cases, the non-invasive stimulation parameters are directed at inducing beta waves in human subjects. In some cases, the non-invasive stimulation parameters are directed at inducing gamma waves in human subjects.

In some cases, light levels and hue are adjusted to avoid fatiguing the subject. In some cases, light levels and hue are adjusted to provide motivation to the subject. In some cases, parameters to each ear or eye are adjusted in a similar manner. In some cases, parameters to each ear or eye are adjusted differently. In an exemplary embodiment, audio, and visual parameters such as tone and hue are varied to provide engagement or motivation to the subject to continue applying the stimulus or monitoring.

### Administering the Non-invasive Sensory Stimulus

Stimulus exposure: session duration, session frequency, and regimen duration

In some cases, administering comprises a stimulus exposure. In some cases, the stimulus exposure comprises a session duration, a session frequency, a regimen duration, or a combination thereof. In some cases, the stimulus exposure may comprise more than one regimen duration. In some cases, administering may be performed continuously for a session duration. In some cases, the session duration may be between 10 minutes and 2 hours. In some cases, the session duration may be at least 1, 2, 3, 4, 5, 10, 15, 20, 25, 30, 35, 40, 45, 50, 55, or 60 minutes. In some cases, the session duration may be at least 1, 2, 3, 4, 5, 6, 7, 8, 9, 10, 11, 12, 13, 14, 15, 16, 17, 18, 19, 20, 21, 22, 23, or 24 hours. In some cases, the session duration may be at least 1, 2, 3, 4, 5, 6, or 7 days. In some cases, the session duration may be at most 1, 2, 3, 4, 5, 10, 15, 20, 25, 30, 35, 40, 45, 50, 55, or 60 minutes. In some cases, the session duration may be at most 1, 2, 3, 4, 5, 6, 7, 8, 9, 10, 11, 12, 13, 14, 15, 16, 17, 18, 19, 20, 21, 22, 23, or 24 hours. In some cases, the session duration may be at most 1, 2, 3, 4, 5, 6, or 7 days.

In some cases, the session frequency may occur at least once, at least twice, at least 3 times, at least 4 times, or at least 5 times per day. In some cases, the session frequency may occur at least once, at least twice, at least 3 times, at least 4 times, at least 5 times, at least 6 times, or at least 7 times per week. In some cases, the session frequency may occur at least once, at least twice, at least 3 times, at least 4 times, or at least 5 times, at least 8 times, at least 10 times, at least 20 times, at least 28 times, at least 30 times, or at least 31 times per month. In some cases, the session frequency may occur at least once, at least twice, at least 3 times, at least 4 times, or at least 5 times, at least 10 times, at least 20 times, at least 30 times, at least 50 times, at least 100 times, at least 150 times, at least 200 times, at least 300 times, at least 365 times, at least 400 times, at least 500 times, at least 600 times, at least 700 times, at least 800 times, at least 900 times, or at least one thousand times per year.

The regimen duration refers to the length of time over which all session durations and session frequencies for the administering of the non-invasive stimulus occur. For example, a regimen duration can refer to a total length of an experimental study period, a total length of a therapeutic treatment, or a total length of a diagnostic period. In some cases, the regimen duration comprises at least one day, at least one week, at least one month, at least 3 months, at least 4 months, at least 5 months, at least 6 months, at least 8 months, at least 9 months, at least 10 months, at least 11 months, at least one year, at least 2 years, at least 3 years, at least 4 years, at least 5 years, at least 10 years, at least 15 years, or at least 20 years. In some cases, the regimen duration comprises the reminder of the life expectancy of the subject. In some cases, the regimen duration comprises the remainder of the subject's life.

In some cases, the regimen duration and session frequency comprises about once a day for 6 months. In some cases, the regimen duration and session frequency comprises at least about 1, 2, 3, 4, 5, 6, 7, 8, 9, or 10 times a day. In some cases, the regimen duration and session frequency comprises at most about 1, 2, 3, 4, 5, 6, 7, 8, 9, or 10 times a day.

### Neural Stimulation System

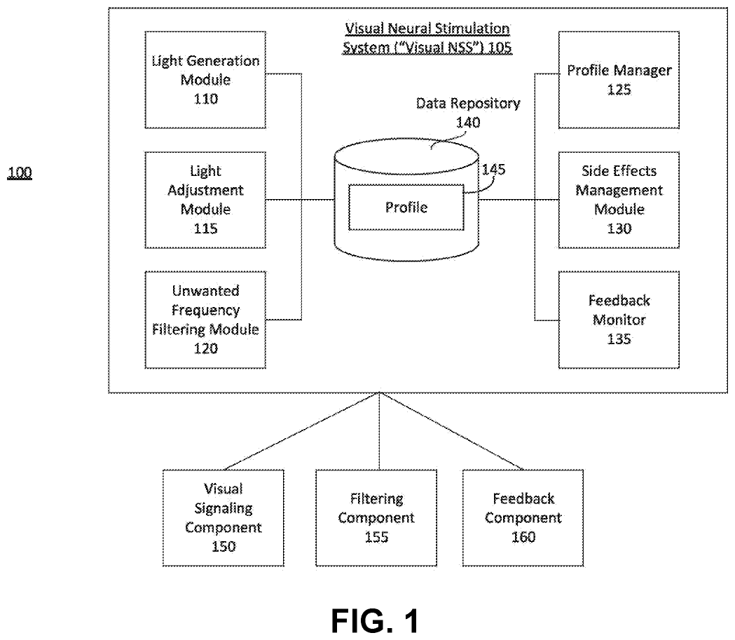

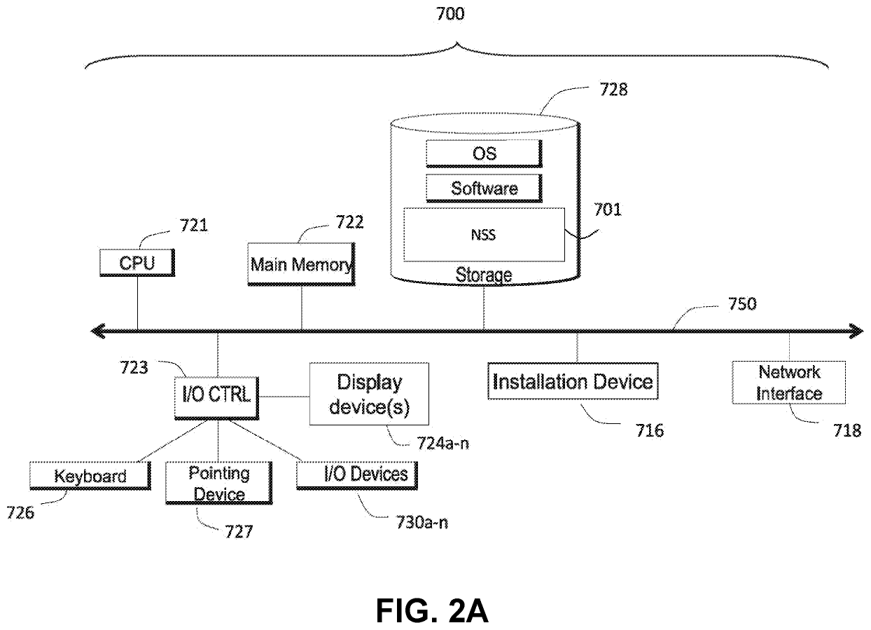

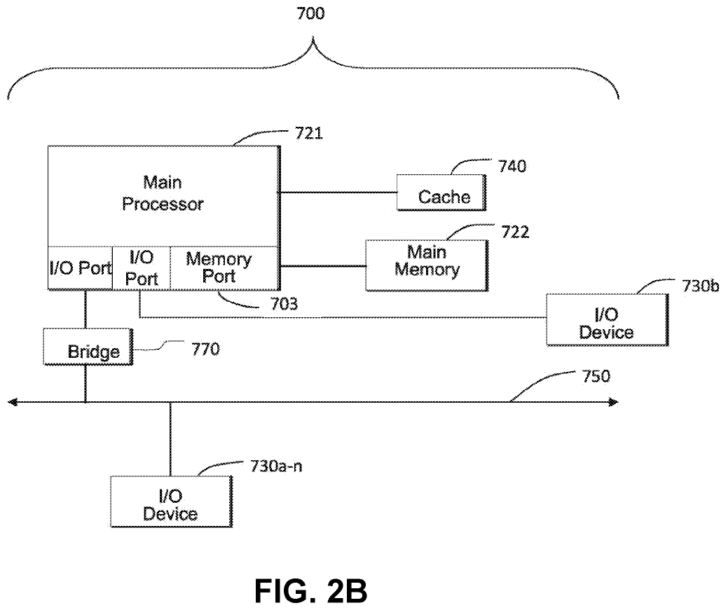

FIG. 1 is a block diagram depicting a system to perform the administration of a non-invasive sensory stimulus in accordance with an embodiment. The system 100 can include a neural stimulation system (“NSS”) 105. The NSS 105 can be referred to as visual NSS 105 or NSS 105. In brief overview, the NSS 105 can include, access, interface with, or otherwise communicate with one or more of a light generation module 110, light adjustment module 115, unwanted frequency filtering module 120, profile manager 125, side effects management module 130, feedback monitor 135, data repository 140, visual signaling component 150, filtering component 155, or feedback component 160. The light generation module 110, light adjustment module 115, unwanted frequency filtering module 120, profile manager 125, side effects management module 130, feedback monitor 135, visual signaling component 150, filtering component 155, or feedback component 160 can each include at least one processing unit or other logic device such as programmable logic array engine, or module configured to communicate with the database repository 150. The light generation module 110, light adjustment module 115, unwanted frequency filtering module 120, profile manager 125, side effects management module 130, feedback monitor 135, visual signaling component 150, filtering component 155, or feedback component 160 can be separate components, a single component, or part of the NSS 105. The system 100 and its components, such as the NSS 105, may include hardware elements, such as one or more processors, logic devices, or circuits. The system 100 and its components, such as the NSS 105, can include one or more hardware or interface component depicted in system 700 in FIG. 2A and FIG. 2B. For example, a component of system 100 can include or execute on one or more processors 721, access storage 728 or memory 722, and communicate via network interface 718.

### Computing Environment

FIGS. 2A and 2B depict block diagrams of a computing device 700. As shown in FIGS. 2A and 2B, each computing device 700 includes a central processing unit 721, and a main memory unit 722. As shown in FIG. 2A, a computing device 700 can include a storage device 728, an installation device 716, a network interface 718, an I/O controller 723, display devices 724a-724n, a keyboard 726 and a pointing device 727, e.g., a mouse. The storage device 728 can include, without limitation, an operating system, software, and software of a neural stimulation system (“NSS”) 701. The NSS 701 can include or refer to one or more of NSS 105, NSS 905, or NSOS 1605. As shown in FIG. 2B, each computing device 700 can also include additional optional elements, e.g., a memory port 703, a bridge 770, one or more input/output devices 730 a-730n (generally referred to using reference numeral 730), and a cache memory 740 in communication with the central processing unit 721.

### Methods of Verifying Administration of a Non-Invasive Sensory Stimulus

In some cases, the administration of a non-invasive sensory stimulus to a subject is confirmed or verified within the subject by evaluating and measuring the non-invasive sensory stimulus. In some cases, the non-invasive sensory stimulus is measured in the brain of the subject. In some cases, the non-invasive sensory stimulus is measured in at least one, at least two, at least three, at least 4, at least 5, at least 8, at least 10, at least 15, at least 20 specific brain regions of the subject. In some cases, the non-invasive sensory stimulus is measured simultaneously in at least one, at least two, at least three, at least 4, at least 5, at least 8, at least 10, at least 15, at least 20 specific brain regions of the subject.

In some cases, the measuring comprises analyzing the brain or a specific brain region of the subject. In some cases, the measuring comprises analyzing the non-invasive sensory stimulus in the brain or a specific brain region of the subject.

In some cases, the specific brain region comprises an entorhinal cortex, a hippocampus, a cerebral cortex, a visual association cortex, an auditory association cortex, a cingulate lobe, an amygdala, a thalamic nucleus, a cingulate lobe, a pons, a brainstem, a cerebellum, a lateral ventricle, an occipital lobe, a parietal lobe, a temporal lobe, a midbrain, a striatum, a basal ganglia, a globus pallidus, a substantia nigra, or any combination thereof.

In some cases, the measuring comprises neuroimaging. In some cases, the measuring comprises neuroimaging of the brain of the subject. In some cases, the measuring comprises neuroimaging of the whole brain of the subject. In some cases, the measuring comprises neuroimaging of the specific brain regions of the subject. In some cases, the measuring comprises neuroimaging of an entorhinal cortex, a hippocampus, a cerebral cortex, a visual association cortex, an auditory association cortex, a cingulate lobe, an amygdala, a thalamic nucleus, a cingulate lobe, a pons, a brainstem, a cerebellum, a lateral ventricle, an occipital lobe, a parietal lobe, a temporal lobe, a midbrain, a striatum, a basal ganglia, a globus pallidus, a substantia nigra, or any combination thereof.

In some cases, the measuring comprises neuroimaging techniques.

In some cases, the neuroimaging comprises magnetic resonance imaging (MRI), computer tomography, positron emission tomography (PET) imaging, diffusion-weighted MRI imaging, or any combination thereof.

### MRI

In some cases, the MRI comprises a T1-weighted MRI. In some cases, the MRI comprises a T2 weighted MRI. In some cases, the MRI comprises a T1-weighted MRI and a T2-weighted MRI. In some cases, the MRI comprises a structural MRI, a T2-weighted MRI, a T2-weighted-fluid-attenuated inversion recovery imaging, a gradient recalled echo T2-weighted MRI, or an arterial spin labeling (ASL) MRI.

In some cases, the MRI comprises a diffusion-weighted MRI. In some cases, the diffusion-weighted MRI comprises free-water imaging (FWI) or diffusion tensor imaging (DTI). In some cases, the MRI comprises a functional MRI (fMRI).

In some cases, the measuring comprises measuring a T1-weighted intensity, a T2-weighted intensity, a T1/T2-weighted ratio, a white matter volume, a gray matter volume, or any combination thereof.

### PET Imaging

In some cases, the PET imaging comprises Electrical readout of neurophysiology (EEG) or Magnetoencephalography (MEG).

In some cases, the measuring comprises an electrical readout of neurophysiology.

In some cases, the electrical readout of neurophysiology comprises an electroencephalogram (EEG) recording, magnetoencephalography (MEG) recording or local field potential.

In some cases, the EEG recording or local field potential measures a feature of the gamma waveform.

In some cases, the measuring comprises measuring two or more features of the gamma waveform.

In some cases, the features of the gamma waveform comprises the amplitude, the frequency, the duration, the frequency distribution, the maximum frequency, the minimum frequency of the gamma waveform, or any combination thereof.

In some cases, the feature of the gamma waveform comprises a measured amplitude of the gamma waveform, a frequency distribution of the gamma waveform, a duration of the gamma waveform, a frequency of the gamma waveform, a neuroanatomical distribution of the gamma waveform in a brain region, a neuroanatomical distribution of the gamma waveform throughout the brain, or any combination thereof.

### Therapeutic Efficacy

In some cases, confirming or perceiving the non-invasive sensory stimulus comprises quantifying a therapeutic efficacy in a subject caused by administration of the non-invasive sensory stimulus to the subject. A therapeutic efficacy comprises a change that reflects, resembles, or indicates a benefit, improvement, or resistance to disease progression in the physiology or neurophysiological of the subject in response to experimental, clinical, or pharmacological intervention or treatment. In some cases, a therapeutic efficacy comprises a change that reflects, resembles, or indicates a benefit, improvement, or resistance to disease progression in the physiology or neurophysiology of the subject. In some cases, a therapeutic efficacy comprises a change that reflects, resembles, or indicates a benefit, improvement, or resistance to disease progression in the physiology or neurophysiology of the subject in response to administration of a non-invasive sensory stimulus. In some cases, therapeutic efficacy in the subject is caused by administering the non-invasive sensory stimulus to the subject.

In some cases, the therapeutic efficacy comprises maintenance of brain volume, cortical thickness, synaptic connectivity, cognitive function, sleep quality, myelination, daily living activities, cerebral blood flow, brain network connectivity, or a combination thereof.

In some cases, the therapeutic efficacy comprises an improvement of brain volume, cortical thickness, synaptic connectivity, cognitive function, sleep quality, myelination, daily living activities, cerebral blood flow, brain network connectivity, cerebral blood flow, brain network connectivity, or a combination thereof.

In some cases, the therapeutic efficacy comprises an increase in cerebral blood flow, brain network connectivity, cortical thickness, brain volume, cognitive function, synaptic connectivity, daily living activities, myelination, or any combination there.

In some cases, the therapeutic efficacy comprises a decrease in brain network connectivity, synaptic connectivity, WASO, or any combination thereof.

In some cases, the therapeutic efficacy further comprises an increase of the amplitude, the frequency, the duration, the frequency distribution of the gamma waveform, or any combination thereof following administration of the non-invasive sensory stimulus compared to the subject prior to administration.

In some cases, the therapeutic efficacy further comprises a decrease of the amplitude, the frequency, the duration, the frequency distribution of the gamma waveform, or any combination thereof following administration of the non-invasive sensory stimulus compared to the subject prior to administration.

In some cases, the therapeutic efficacy further comprises a maintenance or increase of the amplitude, the frequency, the duration, the frequency distribution of the gamma waveform, or any combination thereof following administration of the non-invasive sensory stimulus to the subject compared to the control subject.

In some cases, the therapeutic efficacy comprises a maintenance or increase of the T1-weighted intensity, T2-weighted intensity, T1/T2-weighted ratio, white matter volume, gray matter volume, or any combination thereof compared to before administering the non-invasive sensory stimulus to the subject.

In some cases, the therapeutic efficacy comprises an increase of the T1-weighted intensity, T2-weighted intensity, T1/T2-weighted ratio, white matter volume, gray matter volume, or any combination thereof in the subject compared to the control subject.

In some cases, the therapeutic efficacy comprises a reduction or decrease of the time-activity concentration, the SUV, the SUVR, or any combination thereof in the subject following administration of the non-invasive sensory stimulus compared to the subject prior to administering the non-invasive sensory stimulus.

In some cases, the therapeutic efficacy comprises an increase of the decay constant of the activity concentration in the subject following administration of the non-invasive sensory stimulus compared to the subject prior to administering the non-invasive sensory stimulus.

In some cases, the therapeutic efficacy comprises both: 1) a reduction of the time-activity concentration, the SUVR, or any combination thereof in the subject; and 2) an increase of the decay constant of the activity concentration following administration of the non-invasive sensory stimulus compared to the subject prior to administering the non-invasive sensory stimulus.

In some cases, the therapeutic efficacy comprises a greater difference in the time-activity concentration, the SUV, the SUVR, or any combination thereof in the subject following administration of the non-invasive sensory stimulus compared to the control subject.

In some cases, the therapeutic efficacy comprises both: 1) a smaller difference in the time-activity concentration, the SUV, the SUVR, or any combination thereof; and 2) a larger decay constant of the activity concentration, in the subject following administration of the non-invasive sensory stimulus compared to the control subject.

### Subject

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

In some cases, the human receives an experimental treatment or augmentation.

In some cases, the subject comprises a mammal. In some cases, the subject comprises a non-human primate. In some cases, the subject comprises a human. In some cases, the mammal comprises a non-human primate.

In some cases, the subject is a healthy individual. In some cases, the subject is an individual that does not have or is not suspected of having a neurological disorder or condition. In some cases, the subject does not present or exhibit symptoms of a neurological disorder or condition. In some cases, the subject comprises a subject who is not exposed to the non-invasive sensory stimulus. In some cases, the subject is an individual who exhibits or presents symptoms of a neurological disorder or condition. In some cases, the subject is an individual who has been diagnosed with a neurological disorder or condition. In some cases, the subject is an individual who is suspected of having a neurological disorder or condition. In some cases, the subject is an individual who is at risk of developing a neurological disorder.

In some cases, the subject is an individual that does not have or is not suspected of having any neurological disorder or condition described herein. In some cases, the subject does not present or exhibit symptoms of any neurological disorder or condition described herein. In some cases, the subject is an individual who exhibits or presents symptoms of at least one neurological disorder or condition described herein. In some cases, the subject is an individual who has been diagnosed with a neurological disorder or condition described herein. In some cases, the subject is an individual who is suspected of having one or more of the neurological disorders or conditions described herein. In some cases, the subject is an individual who is at risk of developing a neurological disorder or condition described herein.

In some cases, the mammal comprises a human. In some cases, the human is an elderly human, a child, an adolescent human, or an adult human. In some cases, the human is a human who is physically healthy. In some cases, the human has or is suspected of having a neurological disorder or condition. In some cases, the human has been diagnosed with a neurological disorder or condition.

What is claimed is:

1. A method of confirming or verifying the administration of a non-invasive sensory stimulus to a subject comprising:
a. administering a non-invasive sensory stimulus to a brain of the subject;
b. quantifying the non-invasive sensory stimulus to the subject, thereby verifying that the non-invasive sensory stimulus was administered to the subject.

2. The method of claim 1, wherein the non-invasive sensory stimulus has a gamma frequency.

3. The method of claim 1, wherein the administering of the non-invasive sensory stimulus to the brain of the subject results in an alteration of neural activity in the subject.

4. The method of claim 3, wherein the verifying comprises evaluating the alteration of neural activity after administration of the non-invasive sensory stimulus to the subject.

5. The method of claim 4, wherein the evaluating comprises measuring a gamma waveform in the brain of the subject.

6. The method of claim 5, wherein the measuring comprises neuroimaging.

7. The method of claim 6, wherein the neuroimaging comprises magnetic resonance imaging (MRI), computer tomography, positron emission tomography (PET) imaging, diffusion imaging, function near-infrared spectroscopy, or any combination thereof.

8. The method of claim 7, wherein the MRI comprises a functional MRI (fMRI).

9. The method of claim 5 or 6, wherein the measuring comprises an electrical readout of neurophysiology.

10. The method of claim 9, wherein the electrical readout of neurophysiology comprises an electroencephalogram (EEG) recording, magnetoencephalography (MEG) recording, or local field potential.

11. The method of claim 10, wherein the EEG recording or local field potential measure a feature of the gamma waveform.

12. The method of claim 11, wherein the feature of the gamma waveform comprises the amplitude, the frequency, the duration, the frequency distribution, the maximum frequency, the minimum frequency of the gamma waveform, or any combination thereof.

13. The method of any one of claims 5-11, wherein the measuring comprises quantifying a therapeutic efficacy in the subject caused by administering the non-invasive sensory stimulus in the subject.

14. The method of claim 13, wherein the therapeutic efficacy comprises maintenance of brain volume, cortical thickness, synaptic connectivity, cognitive function, sleep quality, myelination, daily living activities, or a combination thereof.

15. The method of any one of claims 1-14 wherein the non-invasive sensory stimulus comprises a visual stimulus, an auditory stimulus, a tactile stimulus, a vibrotactile stimulus, a peripheral nerve stimulus, or a combination thereof.

16. The method of claim 15, wherein a characteristic of the non-invasive sensory stimulus comprises a stimulus feature or a stimulus exposure.

17. The method of claim 16, wherein the stimulus feature comprises an amplitude or intensity, a frequency, a tone, a color, a luminance, a signal delay, an offset, a duration, a static image, a sinusoidal grating, a sound, a dynamic image, a dynamic sound, or any combination thereof.

18. The method of claim 15, wherein the frequency of the non-invasive sensory stimulus is from about 20 Hz to about 100 Hz.

19. The method of claim 18, wherein the frequency of the non-invasive sensory stimulus is about 40 Hz.

20. The method of claim 16, wherein the stimulus exposure comprises a session duration of at least 15 minutes, at least 30 minutes, at least 45 minutes, or at least one hour.

21. The method of claim 20, wherein the stimulus exposure further comprises a regimen duration of at least one day, one week, one month, three months, six months, nine months, one year, or any combination thereof.

22. The method of claim 20 or 21, wherein the stimulus exposure comprises at least one exposure per day, at least two exposures per day, at least three exposures per day, at least four exposures per day, or at least five exposures per day.

23. The method of claim 4, wherein the evaluating comprises analyzing the brain or a specific brain region of the subject.

24. The method of claim 23, wherein the specific brain region comprises an entorhinal cortex, a hippocampus, a cerebral cortex, a visual association cortex, an auditory association cortex, a cingulate lobe, an amygdala, a thalamic nucleus, a cingulate lobe, a pons, a brainstem, a cerebellum, a lateral ventricle, an occipital lobe, a parietal lobe, a temporal lobe, a midbrain, a striatum, a basal ganglia, a globus pallidus, a substantia nigra, or any combination thereof.

25. The method of any one of claims 13-24, wherein the therapeutic efficacy further comprises a determination that the amplitude, the frequency, the duration, the frequency distribution, the maximum frequency, the minimum frequency of the gamma waveform, or any combination thereof are increased in the subject following administration of the non-invasive sensory stimulus compared to the subject prior to administration.

26. The method of any one of the preceding claims, wherein the subject comprises a mammal.

27. The method of claim 26, wherein the mammal comprises a non-human primate.

28. The method of claim 27, wherein the mammal comprises a human.

29. The method of any one of the preceding claims, wherein the method is carried out by a neural stimulation system (NSS).
